(19) United States
(12) Patent Application Publication
(10) Pub. No.: US 2010/0030067 A1
(43) Pub. Date: Feb. 4, 2010

(54) SYSTEM, METHOD, COMPUTER-READABLE MEDIUM AND USE FOR IMAGING OF TISSUE IN AN ANATOMICAL STRUCTURE

(75) Inventors: Michael Cornelis Van Beek, Eindhoven (NL); Martinus Bernardus Van Der Mark, Eindhoven (NL); Levinus Pieter Bakker, Eindhoven (NL); Rene Van Den Ham, Eindhoven (NL); Bernardus Hendrikus Wilhelmus Hendriks, Eindhoven (NL); Ralf Hoffmann, Eindhoven (NL); Nijs Cornelis Van Der Vaart, Eindhoven (NL); Marjolein Van Der Voort, Eindhoven (NL)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., EINDHOVEN (NL)

(21) Appl. No.: 12/515,297

(22) PCT Filed: Nov. 14, 2007
(86) PCT No.: PCT/IB07/54626

§ 371 Date: May 18, 2009

(30) Foreign Application Priority Data
Nov. 21, 2006 (EP) 06124433.1

(51) Int. Cl. A61B 5/05; A61B 8/14

(52) U.S. Cl. 600/425; 600/443

(57) ABSTRACT

A system for imaging of prostate cancer in a prostate in vivo is provided. The system utilizes Diffuse Optical Tomography (DOT) for creating a 3D image for the detection of suspicious prostate tissue. The DOT image may be used to guide the biopsy, thereby reducing the number of false negatives. A method, computer-readable medium and use are also provided.

### FIELD OF THE INVENTION

This invention pertains in general to the field of medical imaging. More particularly the invention relates to imaging of prostate cancer in vivo.

### BACKGROUND OF THE INVENTION

Prostate cancer is the most common cancer excluding skin cancer in men. The American Cancer Society, ACS, estimates that about 232,090 new cases of prostate cancer will be diagnosed in the United States and 30,350 men will die of this disease in 2005. The ACS estimates that a male in the US has a 1 in 6 risk of developing prostate cancer during his lifetime.

There are several tests are available for detection of prostate cancer, such as, Prostate-specific antigen (PSA) blood test, Digital rectal exam (DRE), Transrectal ultrasound (TRUS) and Core needle biopsy. PSA, DRE and TRUS all have limited sensitivity and/or specificity to prostate cancer. PSA is mainly used to estimate the risk of having prostate cancer and with DRE only palpable lesions close to the rectal wall may be detected, depending on size and shape etc. The diagnosis of prostate cancer is usually performed using a biopsy in which a small sample of prostate tissue is removed and examined under a microscope. The main method for taking a prostate biopsy is a core needle biopsy using TRUS for guidance. The biopsy is required to diagnose and stage prostate cancer. If a biopsy is taken from a tumor, the pathologist may diagnose cancer with a very high accuracy. The problem, however, is to take a biopsy from the right tissue volume. At the moment TRUS is used as an imaging modality to image diseased tissue. The TRUS systems may also be used to guide a biopsy from the diseased tissue volume. In some cases it is possible to recognize lesions using TRUS, however in many cases no lesions are visible, and in these cases TRUS may only be used to determine the position and size of the prostate. Since the position of the lesion is not known, multiple biopsies, typically between 6 and 13, are taken randomly, in an attempt to encounter at least one of the present tumor lesions. Obviously, this procedure leads to numerous false negatives.

EP 1 559 363 A2 discloses a system combining optical imaging technologies with anatomical imaging technologies (e.g. MR, ultrasound). The system can be used for image guidance that may include guiding a biopsy. A drawback of the system is that the optical imaging technology presented, i.e. fluorescence imaging, therein only has a penetration depth of approximately 1-2 mm into the investigated tissue, limited by the strong scattered of light. Hence, lesions located deeper than 1 mm from the surface of the investigated tissue may not be detected using EP 1 559 363 A2.

Hence an improved system, method, computer-readable medium, and use would be advantageous allowing for enhanced imaging resolution, increased detection of diseased tissue, imaging penetration depth, flexibility, cost effectiveness, and less strain to affected subjects,

### SUMMARY OF THE INVENTION

Accordingly, the present invention preferably seeks to mitigate, alleviate or eliminate one or more of the above-identified deficiencies in the art and disadvantages singly or in any combination and solves at least the above-mentioned problems by providing a system, method, computer-readable medium, and use according to the appended patent claims.

According to one aspect of the invention, a system for imaging of prostate cancer in a prostate in vivo is provided. The system comprises at least three units selected from: an electromagnetic radiation source, and a detector unit, forming a plurality of electromagnetic radiation paths, wherein the electromagnetic radiation source is configured to emit incident electromagnetic radiation on the prostate, and the detector unit is configured to receive the electromagnetic radiation, wherein the electromagnetic radiation has been scattered multiple times in the prostate, the system further comprising: an image reconstruction unit for reconstructing a Diffuse Optical Tomography (DOT) image dataset of the prostate based on the received scattered electromagnetic radiation by the at least one detector unit; and a discrimination unit for discriminating between healthy and diseased tissue based on information in the image dataset.

According to another aspect of the invention, a method for imaging of prostate cancer in a prostate in vivo is provided. The method comprises emitting incident electromagnetic radiation on the prostate, receiving the electromagnetic radiation, wherein the electromagnetic radiation has been scattered multiple times in the prostate, wherein the emitting incident electromagnetic radiation and the receiving the electromagnetic radiation forming plurality of electromagnetic radiation paths, the method further comprising reconstructing a Diffuse Optical Tomography image dataset of the prostate based on the received scattered electromagnetic radiation, and discriminating between healthy and diseased tissue based on information in the image dataset.

According to yet another aspect of the invention, a computer readable medium having embodied thereon a computer-program for processing by a computer for imaging of prostate cancer in a prostate in vivo is provided. The computer program comprises an emitting code segment for emitting incident electromagnetic radiation on the prostate, a receiving code segment for receiving the electromagnetic radiation, wherein the electromagnetic radiation has been scattered multiple times in the prostate, wherein the emitting incident electromagnetic radiation and the receiving the electromagnetic radiation forming plurality of electromagnetic radiation paths, the computer program further comprising a reconstruction code segment for reconstructing a Diffuse Optical Tomography image dataset of the prostate based on the received scattered electromagnetic radiation, and a discrimination code segment for discriminating between healthy and diseased tissue based on information in the image dataset.

According to another aspect of the invention a use of the system according to any of the claims 1-9 for locating and diagnosing a lesion in a tissue in an anatomical structure in vivo is provided.

According to a further aspect of the invention a use of the system according to any of the claims 1-9 for guiding a biopsy of a lesion in a tissue in an anatomical structure in vivo is provided.

According to yet another aspect of the invention a use of DOT for diagnosing prostate cancer in vivo is provided.

Embodiments of the present invention pertain to the use of Diffuse Optical Tomography (DOT) for creating a 3D image for the detection of suspicious prostate tissue. The DOT image may be used to guide the biopsy, thereby reducing the number of false negatives.

### BRIEF DESCRIPTION OF THE DRAWINGS

These and other aspects, features and advantages of which the invention is capable of will be apparent and elucidated from the following description of embodiments of the present invention, reference being made to the accompanying drawings, in which

FIG. 1 is a schematic illustration of a system according to an embodiment;

FIG. 2 is an illustration showing a system according to an embodiment;

FIG. 3 is a schematic illustration of a method according to an embodiment; and

FIG. 4 is a schematic illustration of a computer-readable medium according to an embodiment.

### DETAILED DESCRIPTION OF THE EMBODIMENTS

Several embodiments of the present invention will be described in more detail below with reference to the accompanying drawings in order for those skilled in the art to be able to carry out the invention. The invention may, however, be embodied in many different forms and should not be construed as limited to the embodiments set forth herein. Rather, these embodiments are provided so that this disclosure will be thorough and complete, and will fully convey the scope of the invention to those skilled in the art. The embodiments do not limit the invention, but the invention is only limited by the appended patent claims. Furthermore, the terminology used in the detailed description of the particular embodiments illustrated in the accompanying drawings is not intended to be limiting of the invention.

The following description focuses on embodiments of the present invention applicable to an imaging system and in particular to an imaging system for guiding a tissue biopsy.

Diffuse optical tomography (DOT) is an optical imaging technique that can be used for imaging inside a strongly scattering object, such as in tissue. Due to the strong scattering and absorption it is not possible to make a direct optical image of the interior of an organ. To solve this, the tissue or the organ is illuminated from one or more positions and the diffuse transmitted or reflected electromagnetic radiation is detected at one or more position. From the attenuation between different source-detector pairs the optical properties inside the organ are calculated. Often near-infrared light (NIR) is used because this has a relatively deep penetration depth in biological tissue. For DOT imaging it is beneficial that multiple electromagnetic radiation paths are measured and this requires multiple electromagnetic radiation sources and/or multiple detectors.

The present invention utilizes the technology referred to as Diffuse Optical Tomography, DOT, to image tissue in vivo, such as the prostate. In diffuse optical tomography the intrinsic absorption and scattering properties of tissue may be determined. In the near infrared region the absorption properties are strongly dominated by blood, water and lipids. Therefore in absorption DOT a 3D image dataset of the blood content, the oxygen saturation, and the water concentration of the lipid concentration may be obtained. Additionally a 3D image dataset of the scattering properties may be obtained. As the absorption and scattering properties of tissue are different for malignant and healthy tissue, it is possible to distinguish between malignant and healthy tissue in the created 3D map.

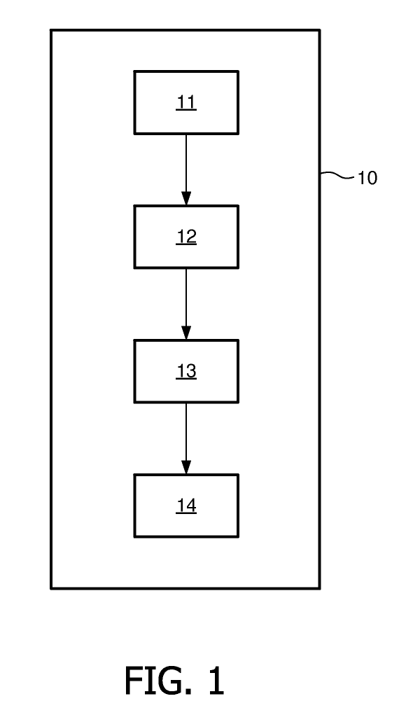

In an embodiment, according to FIG. 1, a system for imaging of tissue in an anatomical structure in vivo is provided. The system comprises at least two electromagnetic radiation sources 11 for emitting incident electromagnetic radiation on the anatomical structure. As the electromagnetic radiation propagates through the anatomical structure it is scattered and partially absorbed in the tissue due to the optical characteristics in the tissue. Different tissue has different optical characteristics and hence the electromagnetic radiation scatters differently depending on the tissue type. The system further comprises at least two detector units 12 for receiving the scattered electromagnetic radiation.

Throughout this specification the system contain at least one source and two or more detectors or the system contains at least one detector and two or more sources. In this way it is possible to measure at least two different electromagnetic radiation paths through the tissue.

Furthermore, it is appreciated that one electromagnetic radiation source that is used to emit electromagnetic radiation at different positions, e.g. a retractable fibre, is considered to be referred to as multiple electromagnetic radiation sources.

Furthermore, an image reconstruction unit 13 is comprised in the system for reconstructing a 3D Diffuse Optical Tomography image of the tissue based on the received scattered electromagnetic radiation received by the two detector units 12. The image contains information of different tissue type and the location of the different tissue types may be calculated from the image. Accordingly, the system may be used to distinguish between healthy and diseased tissue in vivo.

In an embodiment the detected tissue type is characterized as healthy and diseased tissue, such as healthy prostate cells and malignant prostate cells, respectively.

### Diffuse Optical Imaging Modes

The following section describes different ways to perform DOT. All modes require dedicated hardware, software and image reconstruction algorithms.

In an embodiment the system operates utilizing the steady-state domain, i.e. the system measurements, calculations and reconstruction are performed in the steady-state domain, which is also referred to as continuous wave DOT. An advantage of steady-state domain technique is a simple and fairly inexpensive detection system and that low-noise detection electronics may be used for limited cost. However, using a single wavelength only the attenuation, which is a function of the product of absorption and scattering, may be determined using the steady state domain.

In an embodiment the system operates utilizing the time domain, i.e. the system measurements, calculations and reconstruction are performed in the time domain. An advantage of the time domain is that the absorption and scattering properties of the tissue may be distinguished.

In an embodiment the system operates utilizing the frequency domain, i.e. the system measurements, calculations and reconstruction are performed in the frequency domain, which is also referred to as diffuse photon density waves. An advantage for the frequency domain is, in similarity with the time domain, that the absorption and scattering properties of the tissue may be distinguished.

Each of the imaging techniques may be used in two modes, absorption mode that is also referred to as attenuation mode, and fluorescence mode. In the absorption mode the wavelength of the incident electromagnetic radiation and the detected electromagnetic radiation is equal. By using the absorption mode the absorption and scattering properties of tissue are measured, e.g. by measuring attenuation between all source-detector pairs and use image reconstruction.

In an embodiment using absorption mode, the electromagnetic radiation sources emit electromagnetic radiation comprising multiple wavelengths, and the detectors have capability of receiving the multiple wavelengths. The image reconstruction unit uses the spectral information received by the detectors for reconstructing a corresponding 3D image. With multi-wavelength DOT, which is also referred to as spectroscopic DOT, the concentrations of the four near infrared chromophores in tissue may be determined: oxy-hemoglobin, deoxy-hemoglobin, water and lipids.

Alternatively the electromagnetic radiation sources emit electromagnetic radiation that excites the electrons in the atoms of the tissue to a higher energy state. When the electrons returns to a lower energy state the excess energy will be in the form of fluorescence light. Hence the detector unit may be used in the fluorescence mode. In this case filters are used to block the excitation light. The detected fluorescence may be auto-fluorescence from the tissue or fluorescence from an exogenous contrast agent. The detected fluorescence signal depends on the concentration and distribution of the fluorophores and on the scattering and absorption properties of the tissue. Advantages of fluorescence measurements with respect to absorption measurements include: lower background and higher contrast.

In an embodiment the electromagnetic radiation source emits electromagnetic radiation of a single wavelength, i.e. the electromagnetic radiation source having a narrow wavelength spectrum, such as a laser.

In an embodiment auto-fluorescence is used to image the tissue. Using auto-fluorescence no contrast agent injected and the tissue to be imaged, such as the prostate, is illuminated with electromagnetic radiation from a specific excitation wavelength. Fluorescence light in the form of auto-fluorescence is detected and the excitation light is suppressed by filters in the detection path.

In an embodiment a fluorescent contrast agent is injected and the tissue to be imaged, such as the prostate, is illuminated with electromagnetic radiation from a specific excitation wavelength. Fluorescence light is detected and the excitation light is suppressed by filters in the detection path.

### Image Reconstruction

In an embodiment the image calculation utilizes an image reconstruction algorithm for obtaining the resulting 3D image of the tissue. Several known image reconstruction algorithms may be used, such as, but not limited to, (filtered) back-projection, and finite element modeling (FEM).

The image reconstruction unit may be any unit normally used for performing the involved tasks, e.g. a hardware, such as a processor with a memory. The processor may be any of variety of processors, such as Intel or AMD processors, CPUs, microprocessors, Programmable Intelligent Computer (PIC) microcontrollers, Digital Signal Processors (DSP), etc. However, the scope of the invention is not limited to these specific processors. The memory may be any memory capable of storing information, such as Random Access Memories (RAM) such as, Double Density RAM (DDR, DDR2), Single Density RAM (SDRAM), Static RAM (SRAM), Dynamic RAM (DRAM), Video RAM (VRAM), etc. The memory may also be a FLASH memory such as a USB, Compact Flash, SmartMedia, MMC memory, MemoryStick, SD Card, MiniSD, MicroSD, xD Card, TransFlash, and MicroDrive memory etc. However, the scope of the invention is not limited to these specific memories.

In an embodiment the apparatus is comprised in a medical workstation or medical system, such as a Computed Tomography (CT) system, Magnetic Resonance Imaging (MRI) System or Ultrasound Imaging (US) system.

### Detector Unit

In an embodiment the detector unit is a photo detector capable of detecting the total amount of electromagnetic radiation incident on the detector. An example of such a detector is a silicon photodiode.

In an embodiment the detector unit is a spectrophotometer capable of detecting multiple wavelengths from the received scattered electromagnetic radiation.

In another embodiment the detector unit comprises one or more detector arrays.

In a further embodiment the detector comprises a combination of optics and a detector chip. If the detector chip is a monochrome detector chip, it has not capability of identifying the wavelengths of the received electromagnetic radiation. In such a case the optics may e.g. be a lens system, grating or a prism to provide refraction of the received electromagnetic radiation before hitting the detector chip in order to being able to identify the wavelength spectrum of the received electromagnetic radiation and hence provide information to the image reconstruction unit regarding possible tissue type etc.

Several detector chips may be used, such as, but not limited to, Charged Coupled Device CCD chips or Complimentary Metal-Oxide Semiconductor CMOS chips. Color CCD and CMOS chips, are equally possible within the scope of the invention. An alternative embodiment to obtain spectral information is to use a wavelength independent detector (silicon photodiode) and illuminate the tissue sequentially with electromagnetic radiation from different wavelengths.

### Electromagnetic Radiation Source

In an embodiment the electromagnetic radiation source emits electromagnetic radiation comprising a single wavelength or electromagnetic radiation from a small wavelength region centered around the single wavelength.

It is also possible to use a broadband electromagnetic radiation source and measure the received broadband electromagnetic radiation using a detector unit. In an embodiment the electromagnetic radiation source emits electromagnetic radiation comprising multiple wavelengths. Examples of such electromagnetic radiation sources are, but not limited to: incandescent light bulbs, which emit only around 10% of their energy as visible light and the remainder as infrared light, light-emitting diodes, gas discharge lamps, such as neon lamps and neon signs, mercury-vapor lamps, and lasers etc.

### Probes

In an embodiment the system comprises a transrectal or transurethral probe, in which all of the electromagnetic radiation sources and detectors of the system is comprised. Accordingly, the single probe contains one or more electromagnetic radiation sources and one or more detectors.

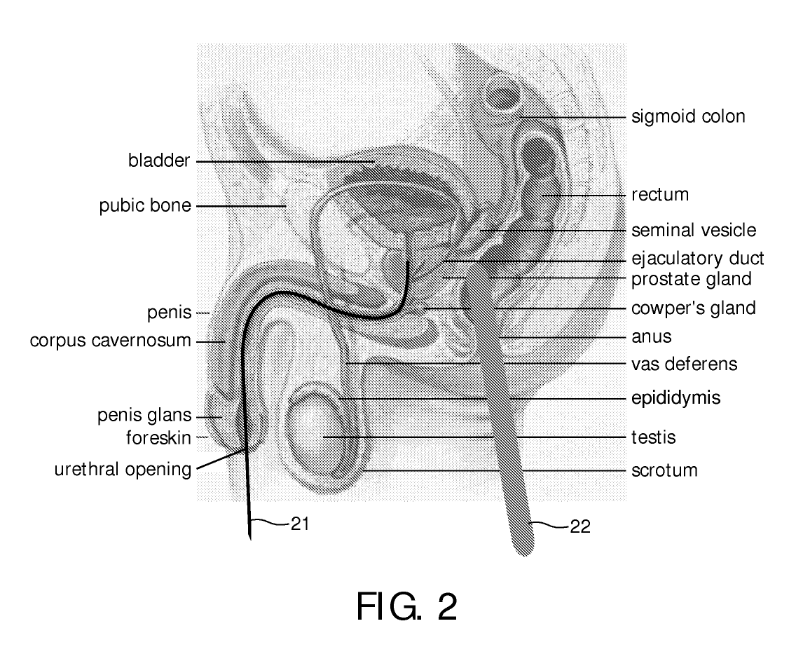

In an embodiment the system comprise both a transrectal probe and a transurethral probe. The transurethral probe comprises one or more of the electromagnetic radiation sources. In use the transurethral probe is placed in the urethra in the vicinity of the prostate. The transrectal probe comprises one or more detectors for receiving the electromagnetic radiation from the electromagnetic radiation sources of the transurethral probe. In use the transrectal probe is placed in the rectum in the vicinity from the prostate. FIG. 2 illustrates the position of the transurethral 21 and transrectal probe 22 in use according to an embodiment.

In some embodiments the transrectal and transurethral probe are positioned in such a way that the prostate is located between the probes. More particularly, the probes are positioned such that the emitted electromagnetic radiation from the urethral probe propagates through the prostate and the detectors of the transrectal probe are the positioned to receive the scattered electromagnetic radiation. Using this setup the system will be sensitive to the tissue properties in the prostate, and hence disturbances from surrounding tissue will be minimal.

In an embodiment the transrectal probe further comprises electromagnetic radiation sources for emitting electromagnetic radiation scattered in the prostate.

In an embodiment the transurethral probe further comprises at least one detector unit for receiving electromagnetic radiation scattered in the prostate.

In an embodiment a bladder probe is comprised in the system. The bladder probe has the shape of an umbrella that may be unfolded inside the bladder. The bladder may and contain electromagnetic radiation sources and/or detectors. In use the umbrella touches the bottom of the bladder to be as close a possible to the prostate region.

In another embodiment a saddle probe is comprised in the system. The saddle probe has the shape of a saddle and in use touches the genital area and contains sources and or detectors.

In an embodiment a combination of transrectal, transurethral, bladder, or saddle probe is used for imaging of the prostate gland, wherein each probe may contain zero, one or more electromagnetic radiation sources, and zero, one or more detectors.

In an embodiment at least one of the probes contains at least one source and at least one of the probes contains at least one of the detectors.

In an embodiment the image reconstruction unit utilizes DOT as the only imaging technique.

In an embodiment the transurethral probe is a transurethral endoscope.

In another embodiment the transurethral probe is a fiber, wherein the electromagnetic radiation source is located ex vivo.

In an embodiment the transrectal probe is a transrectal endoscope.

In an embodiment the transrectal and/or transurethral probe comprise an ultrasound unit. Whereas DOT is mainly sensitive to the blood concentration and blood oxygenation, the ultrasound unit provides topographic details, such as the boundary of the prostate, the rectal wall, and the needle for a biopsy. Hence, this embodiment may be used to guide a biopsy after the diseased areas of interest has been located using the image reconstruction unit image. For image reconstruction the position of the electromagnetic radiation sources and the detector units with respect to each other has to be known. This is especially a problem if a combination of two endoscopes is used. The ultrasound unit may be used to determine the position and orientation of the probe or probes with respect to each other. If the ultrasound unit is incorporated into the transrectal probe the transurethral endoscope will be clearly visible and inversely. The combination with ultrasound will improve the resulting image from the imaging reconstruction unit, by overlaying both images or by using anatomical information obtained by US for the image reconstruction of the optical image.

In an embodiment the transrectal and/or transurethral probe comprise a biopsy unit configured to take a biopsy of the prostate. The biopsy unit receives information from the imaging unit regarding the exact location of the tissue type of interest, such as the diseased tissue. This embodiment has the advantage that the biopsy may be performed while imaging the tissue. This eliminates problems with repositioning between a dedicated imaging and a dedicated biopsy tool.

In an embodiment the image reconstruction unit is configured to create an image based on both the detector unit information and the ultrasound unit information continuously.

In an embodiment the distance between each electromagnetic radiation source and each detector is between 2 mm to 10 cm. This means that all detected electromagnetic radiation has been scattered multiple times and hence the diffusion approximation may be used in the image reconstruction algorithm. An advantage of Diffuse Optical Tomography over direct imaging is that the imaging depth is increased up to 10 cm compared to direct imaging 1 mm. Hence, tissue types located deeper than 1 mm is possible to detect using this embodiment.

In a practical implementation a transurethral and a transrectal endoscope according to embodiments are used to guide a biopsy of suspicious malignant prostate tissue. The urethral endoscope contains one or more sources and can be a retractable fiber. The rectal endoscope combines one or more detectors for DOT with an US probe. The US is used to determine the position of the urethral probe with respect to the rectal probe.

The system according to some embodiments of the invention may be used for locating and diagnosing lesions in the human body in vivo. In some applications, once the exact position of a lesion is found a biopsy may be taken from the lesion using e.g. ultra sound techniques for guidance of the biopsy needle. Use of the system drastically reduces the negative biopsy samples compared to currently used “blind sampling” techniques. This reduces patient discomfort and minimizes infections as the number of biopsy samples is reduced. The biopsy may then be analyzed to determine the severity of the lesion. After the biopsy is analyzed a treatment of the lesion area may be performed to cure the patient. In other applications treatment may be performed without the need of a biopsy. Treatment of the lesion may be performed using radiation therapy, chemotherapy etc.

In an embodiment, according to FIG. 1, a system 10 for imaging of prostate cancer in a prostate in vivo is provided. The system comprises at least three units selected from: an electromagnetic radiation source 11, and a detector unit 12, forming a plurality of electromagnetic radiation paths, wherein the electromagnetic radiation source is configured to emit incident electromagnetic radiation on the prostate, and the detector unit is configured to receive the electromagnetic radiation, wherein the electromagnetic radiation has been scattered multiple times in the prostate, the system further comprising: an image reconstruction unit 13 for reconstructing a Diffuse Optical Tomography image dataset of the prostate based on the received scattered electromagnetic radiation by the at least one detector unit; a discrimination unit 14 for discriminating between healthy and diseased tissue based on information in the image dataset.

The discrimination unit may be comprised of a processor and a memory, of the same type as mentioned above regarding an embodiment of the image reconstruction unit, capable of performing image analysis on the Diffuse Optical Tomography image dataset to distinguish between healthy and diseased tissue.

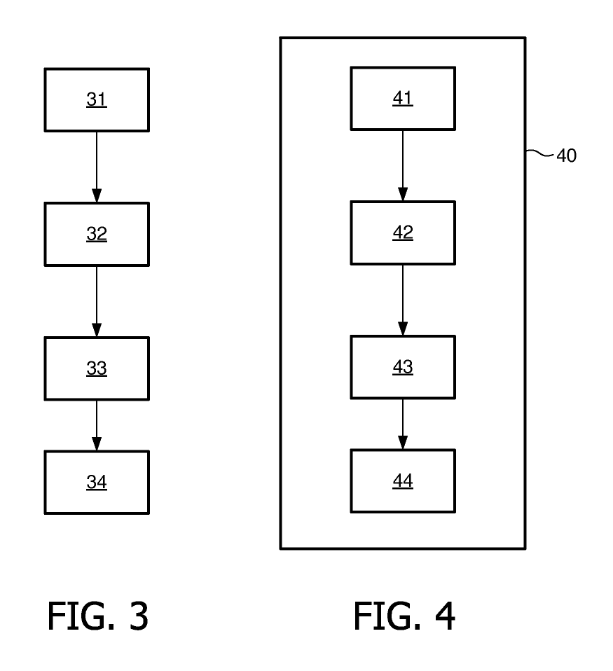

In an embodiment, according to FIG. 3, a method for imaging of tissue in an anatomical structure is provided. The method comprises emitting 31 incident electromagnetic radiation on the prostate, receiving 32 the electromagnetic radiation, wherein the electromagnetic radiation has been scattered multiple times in the prostate, wherein the emitting incident electromagnetic radiation and the receiving the electromagnetic radiation forming plurality of electromagnetic radiation paths, the method further comprising reconstructing 33 a Diffuse Optical Tomography image dataset of the prostate based on the received scattered electromagnetic radiation, and discriminating 34 between healthy and diseased tissue based on information in the image dataset.

In an embodiment the method comprises emitting incident electromagnetic radiation from a transurethral probe on the prostate of a human subject, wherein the transurethral probe is located in the vicinity of the prostate gland, receiving the electromagnetic radiation that has scattered in the prostate by detectors located on a transrectal probe, calculating an image dataset of the tissue based on the received electromagnetic radiation.

In an embodiment a use of the method is provided to locate and diagnose a lesion in the human body in vivo.

In an embodiment, according to FIG. 4, a computer-readable medium 40 having embodied thereon a computer-program for processing by a computer is provided for imaging of tissue in an anatomical structure. The computer program comprises an emitting code segment 41 for emitting incident electromagnetic radiation on the prostate, a receiving code segment 42 for receiving the electromagnetic radiation, wherein the electromagnetic radiation has been scattered multiple times in the prostate, wherein the emitting incident electromagnetic radiation and the receiving the electromagnetic radiation forming plurality of electromagnetic radiation paths, the computer program further comprising a reconstruction code segment 43 for reconstructing a Diffuse Optical Tomography image dataset of the prostate based on the received scattered electromagnetic radiation, and a discrimination code segment 44 for discriminating between healthy and diseased tissue based on information in the image dataset.

In an embodiment the computer-readable medium comprises code segments arranged, when run by an apparatus having computer-processing properties, for performing all of the method steps defined in some embodiments.

In an embodiment the computer-readable medium comprises code segments arranged, when run by an apparatus having computer-processing properties, for performing all of the functions of the system defined in some embodiments.

The invention may be implemented in any suitable form including hardware, software, firmware or any combination of these. The elements and components of an embodiment of the invention may be physically, functionally and logically implemented in any suitable way. Indeed, the functionality may be implemented in a single unit, in a plurality of units or as part of other functional units. As such, the invention may be implemented in a single unit, or may be physically and functionally distributed between different units and processors.

Although the present invention has been described above with reference to specific embodiments, it is not intended to be limited to the specific form set forth herein. Rather, the invention is limited only by the accompanying claims.

In the claims, the term “comprises/comprising” does not exclude the presence of other elements or steps. Furthermore, although individually listed, a plurality of means, elements or method steps may be implemented by e.g. a single unit or processor. Additionally, although individual features may be included in different claims, these may possibly advantageously be combined, and the inclusion in different claims does not imply that a combination of features is not feasible and/or advantageous. In addition, singular references do not exclude a plurality. The terms “a”, “an”, “first”, “second” etc do not preclude a plurality. Reference signs in the claims are provided merely as a clarifying example and shall not be construed as limiting the scope of the claims in any way.

1. A system (10) for imaging of prostate cancer in a prostate in vivo, said system comprising:
at least three units selected from: an electromagnetic radiation source (11), and a detector unit (12), forming a plurality of electromagnetic radiation paths,
wherein said electromagnetic radiation source is configured to emit incident electromagnetic radiation on said prostate, and said detector unit is configured to receive said electromagnetic radiation, wherein said electromagnetic radiation has been scattered multiple times in said prostate, said system further comprising:
an image reconstruction unit (13) for reconstructing a Diffuse Optical Tomography image dataset of said prostate based on said received scattered electromagnetic radiation by said at least one detector unit; and
a discrimination unit (14) for discriminating between healthy and diseased tissue based on information in said image dataset.

2. The system according to claim 1, wherein said at least one electromagnetic radiation source and said at least one detector unit is located on either side of the cancer to be imaged.

3. The system according to claim 1, wherein at least one electromagnetic radiation source is comprised in a urethral unit, said urethral unit being suitable for insertion through urethra and in use positioned in the vicinity of the prostate gland.

4. The system according to claim 1, wherein at least one detector unit is comprised in a transrectal unit, said transrectal unit being suitable for rectal insertion through rectum and in use positioned in the vicinity of the prostate gland.

5. The system according to claim 1, wherein said image dataset is a 2D, 3D, or multi-dimensional image dataset.

6. The system according to claim 1 wherein the distance between each electromagnetic radiation source and each detector unit is 2 mm to 10 cm.

7. The system according to claim 1, further comprising an ultrasound unit for providing an ultrasound image dataset of said prostate.

8. The system according to claim 7, wherein said ultrasound unit is integrated in said transrectal unit, and in use configured to provide an ultrasound image dataset of said prostate.

9. The system according to claim 7, wherein said ultrasound image dataset is used to guide a biopsy of said tissue utilizing the information of said Diffuse Optical Tomography image dataset.

10. A method for imaging of prostate cancer in a prostate in vivo, said method comprising
emitting incident electromagnetic radiation on said prostate,
receiving said electromagnetic radiation, wherein said electromagnetic radiation has been scattered multiple times in said prostate, wherein said emitting incident electromagnetic radiation and said receiving said electromagnetic radiation forming plurality of electromagnetic radiation paths, said method further comprising
reconstructing a Diffuse Optical Tomography image dataset of said prostate based on said received scattered electromagnetic radiation, and
discriminating between healthy and diseased tissue based on information in said image dataset.

11. A computer-readable medium (40) having embodied thereon a computer-program for processing by a computer for imaging of prostate cancer in a prostate in vivo, said computer program comprising
an emitting code segment (41) for emitting incident electromagnetic radiation on said prostate,
a receiving code segment (42) for receiving said electromagnetic radiation, wherein said electromagnetic radiation has been scattered multiple times in said prostate, wherein said emitting incident electromagnetic radiation and said receiving said electromagnetic radiation forming plurality of electromagnetic radiation paths, said computer program further comprising
a reconstruction code segment (43) for reconstructing a Diffuse Optical Tomography image dataset of said prostate based on said received scattered electromagnetic radiation, and
a discrimination code segment (44) for discriminating between healthy and diseased tissue based on information in said image dataset.

12. The computer-readable medium (40) having embodied thereon a computer-program for processing by a computer for imaging of prostate cancer in a prostate in vivo, said computer program comprising
an emitting code segment (41) for emitting incident electromagnetic radiation on said prostate,
a receiving code segment (42) for receiving said electromagnetic radiation, wherein said electromagnetic radiation has been scattered multiple times in said prostate, wherein said emitting incident electromagnetic radiation and said receiving said electromagnetic radiation forming plurality of electromagnetic radiation paths, said computer program further comprising
a reconstruction code segment (43) for reconstructing a Diffuse Optical Tomography image dataset of said prostate based on said received scattered electromagnetic radiation, and
a discrimination code segment (44) for discriminating between healthy and diseased tissue based on information in said image dataset.
comprising code segments arranged, when run by an apparatus having computer-processing properties, for performing all of the method steps defined in claim 10.

13. Use of the system according to claim 1 for locating and diagnosing a lesion in a tissue in an anatomical structure in vivo.

14. Use of the system according to claim 1 for guiding a biopsy of a lesion in a tissue in an anatomical structure in vivo.

15. Use of Diffuse Optical Tomography for diagnosing prostate cancer in vivo.
